(19) United States
(12) Patent Application Publication
(10) Pub. No.: US 2020/0155038 A1
(43) Pub. Date: May 21, 2020

(54) THERAPY MONITORING SYSTEM

(71) Applicant: Massachusetts Institute of Technology, Cambridge, MA (US)

(72) Inventors: Dina Katabi, Boston, MA (US); Zachary Edward Kabelac, Somerville, MA (US); Kreshnik Hoti, Cambridge, MA (US)

(21) Appl. No.: 16/689,716

(22) Filed: Nov. 20, 2019

Related U.S. Application Data
(60) Provisional application No. 62/769,859, filed on Nov. 20, 2018.

(51) Int. Cl. A61B 5/11

(52) U.S. Cl. CPC A61B 5/1104; A61B 5/1113; A61B 5/1116; G01S 13/88; A61B 2503/08; A61B 2505/07; A61B 5/1118

(57) ABSTRACT

A method for monitoring a patient undergoing therapy includes wirelessly tracking a motion of the patient over time to generate a sequence of data representing the motion of the patient over time, processing the sequence of data representing the motion of the patient over time using a biometric parameter extraction module to extract data representing one or more biometric parameters, determining an estimated response of the patient to the therapy based at least in part of the data representing one or more biometric parameters, and providing a characterization of the estimated response of the patient to the therapy to one or more of a caretaker, the patient, or a clinician.

### CROSS-REFERENCES TO RELATED APPLICATIONS

This application claims the benefit of U.S. Provisional Application No. 62/769,859 filed Nov. 20, 2018, the contents of which are incorporated herein by reference.

### BACKGROUND OF THE INVENTION

This invention relates to a therapy monitoring system.

Drug related problems (DRP) are defined as events involving drug therapy that interfere with patients' desired health outcomes. There are various causes for a DRP to occur including adverse drug reactions, unnecessary drug therapy, dosage issues (i.e. dosage being too low or too high, frequency and timing of administration), inappropriate drug selection, use or storage, drug abuse, drug interactions, lack of monitoring, drug non-compliance or non-adherence etc.

As a public health issue, drug non-compliance occurs at every level of the population, but it is particularly associated to patients with chronic disease, polypharmacy and the elderly population group. Non-compliance results in chronic disease progression, increased complications and poorer treatment outcomes. As a result of poor compliance, there is an increased incidence of DRPs as well as mortality. Furthermore, medication non-compliance is associated with higher utilization of health services and hospitalizations. Improving medication compliance is in fact becoming a major frontier in healthcare improvement.

One conventional way to monitor human response to drug therapy to detect DRPs is dependent on patient self-report and health professionals' assessment. This assessment is based on subject's laboratory results, direct physical assessment and information derived from wearable sensors or medical devices such as blood pressure devices, sleep apnea devices, glucometers etc. These solutions require direct contact and/or administration to or by the patient, in order to get information required to make the medical assessment in regard to subject's response to drug therapy.

For example, health professionals acquire human response to drug therapy from various sources and combine it with other factors such as medical conditions (co-morbidities), medications, and patient/caregiver questionnaires. Health professionals leverage their training and experience to interpret the meaning of changes in human response and other factors to determine drug therapy response. Once an outcome or anomaly is suspected or detected by the health professional, available interventions to improve medication compliance range from behavioral and educational interventions to integrated care interventions, patient decision aids, packaging and daily reminders.

### SUMMARY OF THE INVENTION

Recent advancements in technology have enabled wireless and remote dentification of various human biometric parameters using information derived from human radio signal reflections. This technology overcomes a number of limitations associated with body contact or privacy issues related to video-based technologies or solutions based on sensor wearing.

Aspects described herein track human response to drug therapy through wireless sensing technology and provide alerts and logging to mitigate any drug related problems that may arise from the drug therapy. In some examples, tracking human response means detecting, assessing, and/or monitoring human response to drug therapy. Aspects described herein use wireless sensing to derive biometric parameters (gait, breathing, heart rate, sleep state, location, etc.), which are provided as input to machine learning models that use supervised or unsupervised training methods to configure an artificial intelligence system to output categorical or quantitative output for detecting events or anomalies in biometric parameters indicative of human response to drug therapy. In addition, other patient relevant factors such as medical conditions (co-morbidities), in-clinic tests and assessments, medications and patient/caregiver questionnaires can be fed into the same machine learning model to improve the performance of the model.

In a general aspect, a method for monitoring a patient undergoing therapy includes wirelessly tracking a motion of the patient over time to generate a sequence of data representing the motion of the patient over time, processing the sequence of data representing the motion of the patient over time using a biometric parameter extraction module to extract data representing one or more biometric parameters, determining an estimated response of the patient to the therapy based at least in part of the data representing one or more biometric parameters, and providing a characterization of the estimated response of the patient to the therapy to one or more of a caretaker, the patient, or a clinician.

Aspects may include one or more of the following features.

Wirelessly tracking the motion of the patient over time may include tracking the motion of the patent using a radio frequency-based motion tracking system. The radio frequency-based motion tracking system may include an FMCW radar motion tracking system. The radio frequency-based motion tracking system may be configured to emit radio frequency signals and to receive radio frequency signals.

Wirelessly tracking the motion of the patient over time may include tracking the motion of the patient using reflections of electromagnetic signals from the patient. The wireless tracking of the motion of the patient over time may include tracking the motion of the patient using reflections of ultrasonic signals from the patient.

Extracting one or more biometric parameters from the motion of the patient may include extracting one or more of location, pose, gait, mobility, activity, breathing, heartrate, emotion, and sleep patterns. Determining the estimated response of the patient to the therapy may include processing the one or more biometric parameters using a model configured to determine the estimated response of the patient to the therapy based on the one or more biometric parameters. The model may include an artificial neural network.

Wirelessly tracking the motion of the patient over time may include determining a sequence of observations of a position of the patient over time. Extracting the one or more biometric parameters from the motion of the patient may include determining a progression the one or more biometric parameters over time. Determining the estimated response of the patient to the therapy may include determining that the patient has a subtherapeutic response to the therapy.

Determining the estimated response of the patient to the therapy may include determining that the patient has a toxic response to the therapy. Determining the estimated response of the patient to the therapy may include identifying dose dependent adverse effects to the therapy suffered by the patient. Determining the estimated response of the patient to the therapy may include identifying withdrawal effects of the therapy suffered by the patient.

Determining the estimated response of the patient to the therapy may include identifying one or more of drug dosing changes or alterations, drug dose increase, drug dose decrease, wearing off of a drug, drug initiation or cessation, drug-drug interactions, drug-food interactions, drug abuse, drug misuse, and illicit drug use.

The method may include determining a compliance to the therapy by the patient based at least in part on the estimated response of the patient to the therapy. The therapy may include a single or multiple drug therapy.

In another general aspect, a system for monitoring a patient undergoing therapy includes a wireless sensor for wirelessly tracking a motion of the patient over time to generate a sequence of data representing the motion of the patient over time, a biometric parameter extraction module for processing the sequence of data representing the motion of the patient over time to extract data representing one or more biometric parameters, a therapy response model for determining an estimated response of the patient to the therapy based at least in part of the data representing one or more biometric parameters, and, an output providing a characterization of the estimated response of the patient to the therapy to one or more of a caretaker, the patient, or a clinician.

In another general aspect, software stored in non-transitory form on a machine-readable medium, for monitoring a patient undergoing therapy includes instructions for causing a computing system to wirelessly track a motion of the patient over time to generate a sequence of data representing the motion of the patient over time, process the sequence of data representing the motion of the patient over time using a biometric parameter extraction module to extract data representing one or more biometric parameters, determine an estimated response of the patient to the therapy based at least in part of the data representing one or more biometric parameters, and provide a characterization of the estimated response of the patient to the therapy to one or more of a caretaker, the patient, or a clinician.

Among other advantages the therapy monitory system can monitor a wide variety of physiological signals—pose, gait, mobility, activity, breathing, heartrate, emotion, sleep patterns—passively without requiring the monitored person to wear sensors on her body. As such, it can address the aforementioned needs and challenges for measuring drug related problems, including medication non-compliance. Further, its ability to monitor physiological signals without requiring sensors on the body makes it particularly suitable for older people who tend to be encumbered by wearable technologies.

Other features and advantages of the invention are apparent from the following description, and from the claims.

### BRIEF DESCRIPTION OF THE DRAWINGS

FIG. 1 is a therapy monitoring system.

FIG. 2 is a therapy timeline.

FIG. 3 is a supervised training system.

FIG. 4 is an unsupervised training system.

FIG. 5 is a plot of walking speed for a patient and a spouse over time.

FIG. 6 is a plot of a patient's location in an environment over time.

FIG. 7 is a plot of a nurse's location in the environment of FIG. 6 over time.

### DETAILED DESCRIPTION

### 1 THERAPY MONITORING SYSTEM

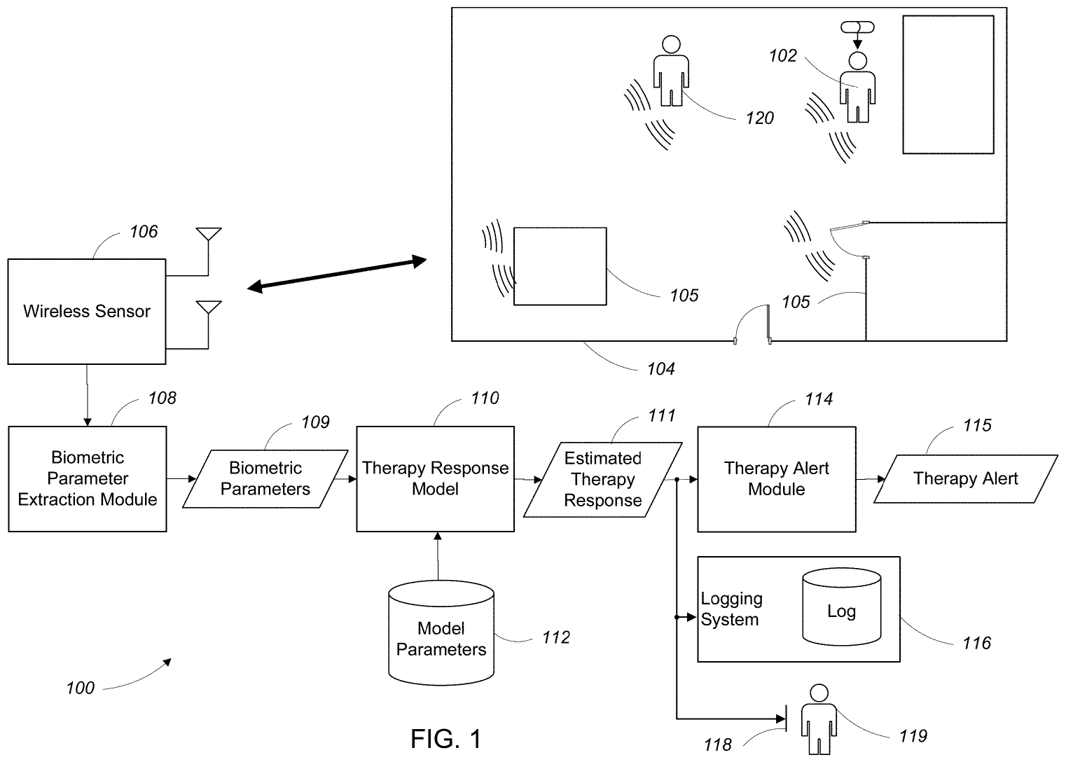

Referring to FIG. 1, a therapy monitoring system 100 includes a wireless sensor 106, a biometric parameter extraction module 108, a therapy response model 110 configured according to model parameters 112, a therapy alert module 114, a logging system 116, and a clinician output 118.

Very generally, the therapy monitoring system 100 is configured to wirelessly monitor biometrics associated with one or more patients 102 undergoing therapy (e.g., a drug therapy) in the environment 104. Based on the wirelessly monitored biometrics, the therapy monitoring system 100 determines response and/or compliance information that is analyzed or logged to determine an efficacy of the therapy and/or to determine whether any of the patients require intervention in their therapy (e.g., a dosage alteration or a compliance assessment).

In one exemplary operation, the wireless sensor 106 repeatedly transmits radio frequency signals into the environment 104 and receives reflections of the transmitted signals from objects 105 in the environment (e.g., furniture and walls) as well as people in the environment (e.g., patients 102 and caregivers 120). The wireless sensor isolates the reflections from the patients 102 from the other reflections and provides the reflections from the patients 102 to the biometric parameter extraction module 108. In some examples, the wireless sensor 106 uses techniques described in one or more of U.S. Pat. No. 9,753,131 and U.S. Patent Publication US 2017/0074980 (both of which are hereby incorporated by reference) to track and distinguish patients and other entities in the environment. In other examples, the wireless sensor uses reflections from electromagnetic or ultrasonic signals to monitor the patient 102.

For each patient 102, the biometric parameter extraction module 108 processes the reflections from the patient 102 (or a part of the patient such as their chest) to determine a time series of locations and/or distances of the patient from the wireless sensor 106. From that time series of locations and/or distances, the biometric parameter extraction module 108 determines one or more biometric parameters 109 such as a heart rate, a breathing rate, a gait, emotional state, a mobility or activity level, or sleep stages, among other biometric parameters 109. In some examples, the biometric parameter extraction module 108 uses the techniques described in one or more of U.S. Patent Publication US 2017/0042432, U.S. Patent Publication US 2017/0311901, U.S. Patent Publication US 2018/0271435, and U.S. Patent Publication US 2019/0188533 (all of which are hereby incorporated by reference) to extract and process the biometric parameters 109.

The biometric parameters 109 extracted by the biometric parameter extraction module 108 are provided to the therapy response model 110, which processes the biometric parameters 109 to determine an estimated therapy response 111. In some examples, the therapy response model 110 is implemented as a computer and/or dedicated hardware implemented artificial neural network, such a deep convolutional or a recurrent neural network. The therapy response model 110 is configured according to the values of model parameters 112, which are obtained in a training phase (described in greater detail below), to determine the estimated therapy response 111 from the biometric parameters 109.

The estimated therapy response is provided to one or more of the therapy alert module 114, the logging system 116, and the clinician output 118.

In some examples, the estimated therapy response 111 (or a representation of the estimated therapy response 111) is provided to the logging system 116 where it is stored for later use by, for example, clinicians or clinical researchers 119.

In some examples, the estimated therapy response 111 (or a representation of the estimated therapy response 111) is directly provided to a clinician 119 by way of the clinician output 118. In such examples, the clinician is able to evaluate the estimated therapy response 111 and determine whether to intervene in the patient's therapy.

In some examples, the therapy alert module 114 processes the estimated therapy response according to one or more rules to determine whether to issue a therapy alert 115 to one or more of the patients 102, the caregiver 120, a clinician 119, or family members of the patient. For example, the clinician 119 and the caregiver 120 may receive a therapy alert if the therapy alert module 114 determines that the patient's current dosage is causing behavior or other response indicative of toxicity. In another example, the caregiver 120, the patient 102, or the patient's family may receive a therapy alert if the therapy alert module 114 indicates that non-compliance with the drug therapy is causing a subtherapeutic response. As is described in greater detail below, any number of rules can be applied by the therapy alert module 114 to determine whether to issue therapy alerts 115 to parties involved with the therapy of a patient 102.

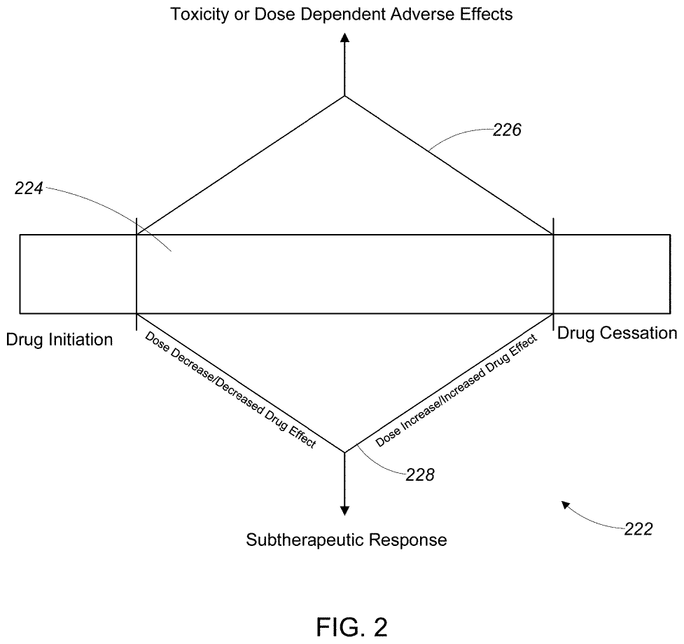

Referring to FIG. 2, in some examples, the one or more rules used by the therapy alert module 114 are derived from key processes related to drug therapy use, which lead to a subject's response to such drug therapy. Some examples of the key processes related to drug therapy use are drug initiation, drug cessation (including drug interruption, re-initiation or intermittent use), dose increase, dose decrease, drug intoxication, drug-drug interaction and drug-food interaction resulting in increased drug effect or decreased drug effect. It is noted that in the case of certain drug groups such as opioids, the abovementioned ‘drug related alerts’ can also arise due to drug abuse, misuse or illicit use.

In FIG. 2, some examples of the key processes during administration of drug therapy to a patient that lead to a subject's drug therapy response are visualized as a timeline 222 extending from therapy initiation to therapy cessation. Along the timeline, there exists a band of appropriate drug therapy effect 224 where a dose and/or a drug effect are appropriate for a patient. A range of doses and/or drug effects that are too great for the patient 226 leads to toxicity and/or dose dependent adverse effects in the patient. A range of doses and/or drug effects that are too little for the patient 228 leads to a subtherapeutic response for the patient. In some examples, the therapy response model 110 generates the estimated therapy response by processing the biometric parameters 109 to estimate where the patient lies in the drug therapy response visualization 222 of FIG. 2. The therapy alert module 114 processes the estimated therapy response 111 according to rules derived from the key processes illustrated in FIG. 2 to determine whether to issue one or more therapy alerts 115.

### 2 TRAINING

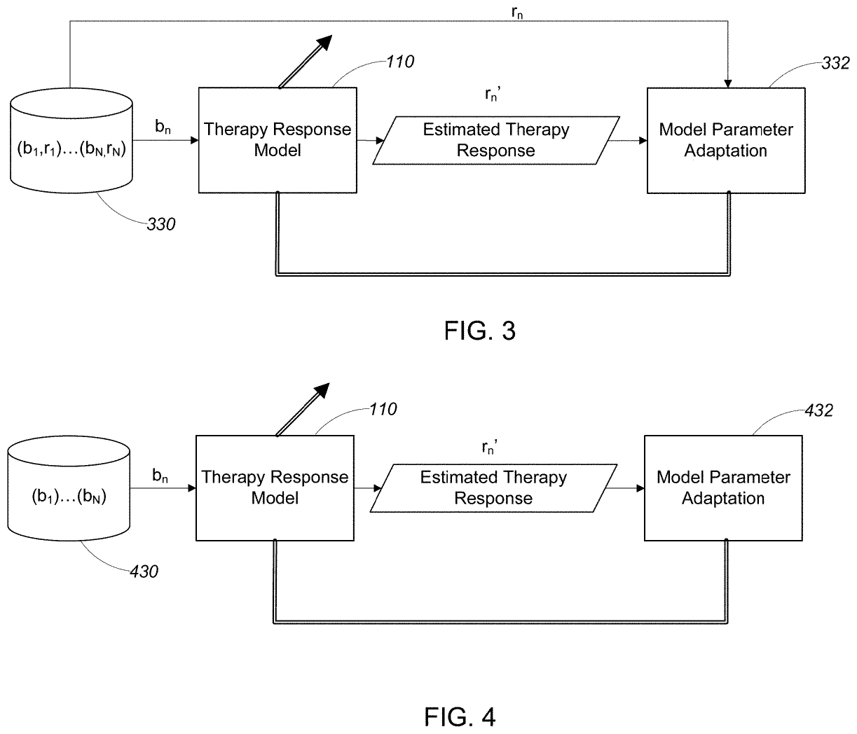

Referring to FIG. 3, in some examples, the therapy response model 110 is trained in a supervised manner using training data 330 including pairs of biometric parameters, bₙ and known therapy responses, rₙ.

To train the therapy response model 110, the biometric parameters, bn are provided to the therapy response model 110, which processes the biometric parameters, bₙ to generate an estimated therapy response, rₙ′. The estimated therapy response rₙ′ is provided to a model parameter adaptation module 332 along with the known therapy response, rₙ.

The model parameter adaptation module 332 processes the estimated therapy response, rₙ′ and the known therapy response, rₙ to generate a set of updated model parameters for configuring the therapy response model. One common way of updating model parameters involves computing/estimating gradients of the combined loss function with respect to the model parameters. For example, backpropagation algorithm for an artificial neural network uses these gradient estimates. Of course, other parameter update schemes can be used.

In some examples, the model parameter adaptation module 332 processes the training data 330 in one or more batches. In other examples, the model parameter adaptation module 332 incrementally updates the model parameters while the runtime system is operating.

Referring to FIG. 4, in some examples, the therapy response model 110 is trained in an unsupervised manner using training data 430 including biometric parameters, bₙ. In some examples, when the therapy response model 110 is trained in an unsupervised manner, it is configured for anomaly detection or to automatically determine to which, of a number of clusters of a population, a particular set of biometric parameters belongs.

To train the therapy response model 110, the biometric parameters, bₙ are provided to the therapy response model 110, which processes the biometric parameters, bₙ to generate an estimated therapy response, rₙ′. The estimated therapy response, rₙ′ is provided to the model parameter adaptation module 432.

The model parameter adaptation module 432 processes the estimated therapy response, rₙ′ a to generate a set of updated model parameters for configuring the therapy response model. The model parameter adaptation module 432 adapts the parameters to identify patterns (e.g., using principal component analysis or cluster analysis) in the biometric parameters, bₙ without requiring that the biometric parameters, rₙ be pre-labeled.

In some examples, the model parameter adaptation module 432 processes the training data 430 in one or more batches. In other examples, the model parameter adaptation module 432 incrementally updates the model parameters while the runtime system is operating.

### 3 EXAMPLES

### 3.1 DETECTION OF OVER SEDATION

In one exemplary use case of the therapy monitoring system, a case of over-sedation due to drug interactions is detected and a therapy alert is issued. For example, if a patient presently taking a medication known to cause sedation begins taking a benzodiazepine medication (known to cause sedation), there is a high probability of a drug interaction that synergistically causes a drug response manifested as over-sedation. The therapy monitoring system wirelessly measures biometric changes such as the patient's sleep stages, sleeping patterns, and time in bed. The measured biometric changes can be provided to a health professional who uses professional expertise and judgement to determine the subject's response to drug therapy. Alternatively, the biometric changes measured can be combined with information from patient's medication (i.e. the presence of benzodiazepine and another sedating drug) and fed into the therapy response model to yield the subtherapeutic response related to excessive sedation. A therapy alert can then be issued to parties involved in the patient's treatment.

### 3.2 MEDICATION IMPACT ON PARKINSON PATIENTS

Parkinson Disease (PD) is a long-term neurodegenerative disease of the central nervous system that primarily affects the motor system. It typically affects people over the age of 60, totaling over 6 million people globally. Both the cause or cure are generally unknown, but it is well accepted that the disease causes nerve cells (neurons) in the brain to gradually break down or die. The loss of neurons reduces the production of dopamine, which causes abnormal brain activity leading to many of the common symptoms of Parkinson Disease. Once the disease is diagnosed, patients and doctors focus on managing and delaying symptoms the best that they can.

The disease causes a number of cognitive and functional symptoms which manifest themselves in a variety of ways that can be monitored by the therapy monitoring system described above. The most common symptoms are tremors, slowed movement also known as bradykinesia, impaired balance and posture, loss of automatic movements, sleep problems, speech and writing changes. Beyond symptoms of the disease, Carbidopa-Levodopa, the most widely used treatment of the last 40 years, leads to the development of long-term complications such as involuntary movements called dyskinesias, and fluctuations in the effectiveness of the medication. When fluctuations occur, a patient can cycle through phases with good responses to medication and reduced PD symptoms (“ON” state), and phases with poor responses to medication and significant PD symptoms (“OFF” state). However, PD symptoms vary widely within and across days so every patient checkup is only a snapshot in time that may or may not reflect the current health of the patient.

The therapy monitoring system 100 described above provides passive patient monitoring to enhance clinical assessments by objectively capturing key processes during administration of drug therapy to a patient at a fine granularity.

The therapy monitoring system 100 monitors the mobility of a patient with PD and their healthy spouse in their home over a 2-month period. As the patient and their spouse move around the home, the system measures their gait speed and/or how quickly they walk. For example, 1,800 gait measurements of the patient and spouse are obtained.

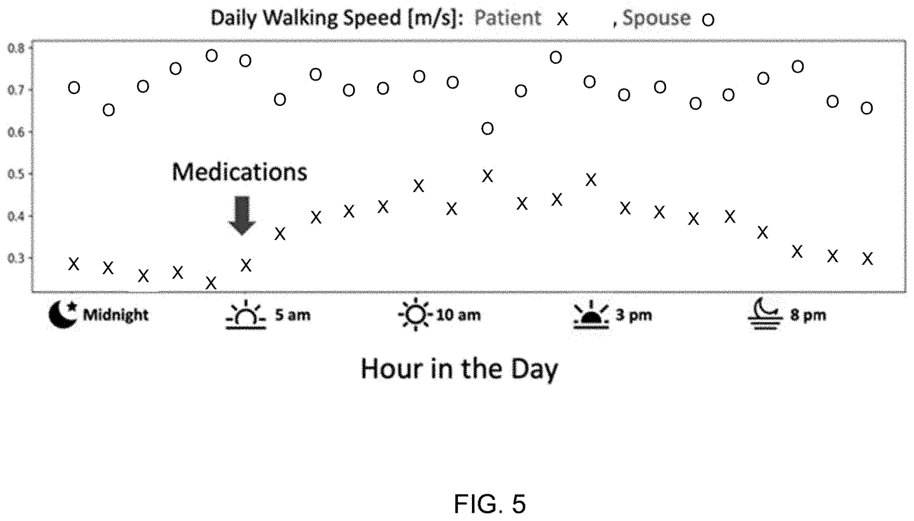

Referring to FIG. 5, the gait speed measurements captured across the 8 weeks are segmented by hour in the day and a median per hour is determined. The X's represent the median gait speed of the patient at each hour and the O's represent the median gait speed of the spouse. It's important to note that the subject and spouse are not awake at all hours of the day, but if they occasionally wake up at 5 am and walk to the bathroom or to drink a glass of water, the system will measure their speed and record it. It then takes the median speed at 5 am across all of the measurements recorded on various days.

The figure shows a significant increase in gait speed between 5 am and 8 am in the morning. This is around the same time that the patient takes their medication hence suggesting a specific patient's response to drug therapy, which in this case could be drug's effects wearing off prior to next dosing. On the other hand, the spouse shows no similar increase in speed indicating that the increase is not due to environmental factors.

### 3.3 MEDICATION ADHERENCE IN ASSISTED LIVING FACILITIES

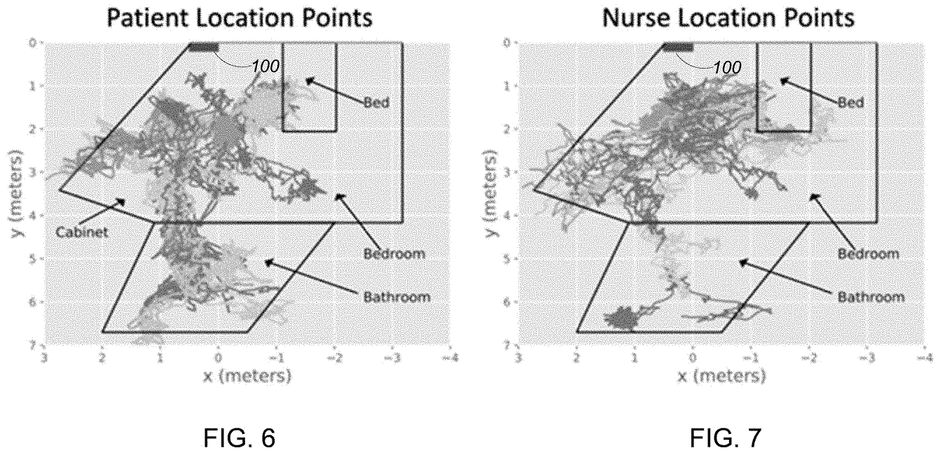

The therapy monitoring system 100 can also be used to analyze localization data of a chronically ill patient in an assisted living facility or at home. Referring to FIGS. 6 and 7, one day of location data gathered from patient's environment is gathered and location points are segmented into to two classes: the patient's locations (shown in FIG. 6) and the nurse's locations (shown in FIG. 7).

With the locations between the patient and the nurse separated, the distinct space usage patterns between the patient and the nurse becomes apparent. For example, the patient goes to the bathroom much more often. This makes sense because the nurse comes for bathroom cleaning only few times a day. From the perspective of patient's response to drug therapy, provided the patient was on diuretic therapy for example, this information would be useful in ascertaining a potential dose dependent adverse effect of patient's diuretic therapy and/or could indicate issues with incorrect drug administration timing. Another observation is that the patient always goes to bed from the left side, while the nurse who helps the patient and makes the bed, approaches the bed from both sides.

Furthermore, it is apparent that the patient never goes to the lower left corner of his bedroom, which is marked as the cabinet in FIG. 6. In this example, the doctor at the facility indicates that the medication cabinet is locked in a closet at that corner and that only the nurse is allowed to give medication to the patient. By tracking when the nurse visits that corner, the patient's medication schedule can be tracked therefore providing useful information on medication compliance.

### 4 ADDITIONAL EXAMPLES

### 4.1 MONITORING OF BREATHING PATTERNS

In some examples, the therapy monitoring system described above monitors and identifies breathing patterns changes to uncover key processes related to a patient undergoing treatment. For example, depressed respiration in patients taking drugs known to cause respiratory depression (such as those experiencing opioid intoxication as a result of opioid use) can be identified, shortness of breath in patients taking drugs known to cause shortness of breath can be identified, wheezing in patients taking drugs known to cause or prevent wheezing can be identified, cough in patients taking drugs known to cause or prevent cough can be identified, and/or subtherapeutic response in cases of bronchodilator subtherapeutic dosing can be identified.

### 4.2 MONITORING OF SLEEP PATTERNS

In some examples, the therapy monitoring system described above monitors and identifies sleeping patterns of a patient to uncover key processes related to a patient undergoing treatment. For example, excessive sedation due to opioid intoxication or excessive dosing can be identified, excessive sedation or dose dependent adverse effect in cases of benzodiazepine intoxication or excessive dosing can be identified, excessive sedation or dose dependent adverse effect in cases of any drug causing drowsiness or sedation can be identified, lack of sleep in cases of opioid withdrawal or subtherapeutic dosing can be identified, subtherapeutic response in cases of benzodiazepine subtherapeutic dosing can be identified, sleep disturbance in cases of dopamine agonists dosing can be identified, and/or sleep disturbance in cases of any drug causing sleep disturbance can be identified.

### 4.3 MONITORING OF MOVEMENT PATTERNS

In some examples, the therapy monitoring system described above monitors and identifies movement patterns of a patient to uncover key processes related to a patient undergoing treatment. For example, the system can indicate itchy skin in cases or dose dependent adverse effect of opioid drugs can be identified, itchy skin in cases of drug related allergic reaction, and/or on/off changes as manifested by tremor or dyskinetic movements in subjects taking anti-Parkinson's drugs.

In some examples, movement patterns are monitored to identify gait changes in subjects taking anti-Parkinson's drugs.

In some examples, movement patterns are monitored to identify tremor in cases of drug initiation, drug intoxication, dose increase, dose dependent adverse effect, drug-drug interaction or drug-food interaction in subjects taking any drug known to cause tremor such as hypoglycemics, antidepressants, opioids, anticonvulsants, etc.

In some examples, movement patterns are monitored to identify dizziness in cases of drug initiation, drug cessation (including drug interruption, re-initiation and intermittent use), drug intoxication, dose increase, dose dependent adverse effect, drug-drug interaction or drug-food interaction in subjects taking any drug whose initiation or dosing is related to causing dizziness such as hypoglycemics, antihypertensives, antipsychotics, anticonvulsants, dopamine agonists, etc.

In some examples, movement patterns are monitored to identify falls or increased risk of falls as a result of drug initiation (including drug interruption, re-initiation and intermittent use), drug intoxication, dose increase, dose dependent adverse effect, drug-drug interaction or drug-food interaction in subjects taking any drug known to cause falls or increased risk of falls such as hypoglycemics, antihypertensives, antipsychotics, anticonvulsants, dopamine agonists, etc.

In some examples, movement patterns are monitored to identify increased use of bathroom and hence polyuria (excessive urination) as a result of a drugs' subtherapeutic effects resulting from subtherapeutic dose, decreased dosing, drug-drug interaction or drug-food interaction in subjects taking hypoglycemic agents.

In some examples, movement patterns are monitored to identify increased use of bathroom and hence polyuria (excessive urination) as a result of a drugs' dose dependent adverse effect resulting from drug initiation (including drug interruption, re-initiation and intermittent use), drug intoxication, dose increase, dose dependent adverse effect, drug-drug interaction or drug-food interaction in subjects taking any drug known to cause increased glucose levels.

In some examples, movement patterns are monitored to identify increased use of bathroom and hence gastrointestinal upset or irritation as a result of a drug intoxication, dose increase (including drug interruption, re-initiation and intermittent use), dose dependent adverse effect, drug-drug interaction or drug-food interaction in cases of subjects taking any drug known to cause gastrointestinal upset or irritation such as hypoglycemics, opioids, serotonergic drugs etc.

In some examples, the therapy monitoring system described above monitors and identifies a heart rate of a patient to uncover key processes related to a patient undergoing treatment. For example, the system can identify arrhythmia in cases of drug initiation, drug cessation (including drug interruption, re-initiation and intermittent use), drug intoxication, dose increase, dose decrease, dose dependent adverse effect, drug-drug interaction or drug-food interaction in subjects taking any drug whose initiation or dosing is related to affecting heart rate such as inotropic agents, beta-blockers, thyroid drugs, antidepressants, antibiotics, corticosteroids etc.

In another example, the system can be used to identify bradycardia in cases of drug initiation (including drug interruption, re-initiation and intermittent use), drug intoxication, dose increase, dose dependent adverse effect, drug-drug interaction or drug-food interaction in subjects taking any drug whose initiation or dosing is related to slowing heart rate such as inotropic agents, beta-blockers, antidepressants, etc.

In another example, the system can be used to identify tachycardia in cases of drug initiation, drug cessation (including drug interruption, re-initiation and intermittent use), drug intoxication, dose increase, dose decrease, dose dependent adverse effect, drug-drug interaction or drug-food interaction in subjects taking any drug whose initiation or dosing is related to affecting heart rate such as inotropic agents, beta-blockers, thyroid drugs, antidepressants, antibiotics, corticosteroids etc.

### 5 APPLICATIONS

One exemplary application of the therapy monitoring system includes the monitoring of patients suffering from or undergoing treatment for diabetes. For example, the therapy monitoring system can monitor a human response to drug therapy indicative of hyperglycemic state and/or a hypoglycemic state.

Another exemplary application of the therapy monitoring system includes the monitoring of patients suffering from or undergoing treatment for hypertension. For example, the therapy monitoring system can monitor a human response to drug therapy indicative of hypotensive state, a hypertensive state, and/or a human response to drug therapy indicative of dry cough in patients taking antihypertensive agents.

Another exemplary application of the therapy monitoring system includes the monitoring of patients suffering from or undergoing treatment for arrhythmias. For example, the therapy monitoring system can monitor a human response to drug therapy indicative of heart rate variation, bradycardia, and/or tachycardia.

Another exemplary application of the therapy monitoring system includes the monitoring of patients suffering from or undergoing treatment for opioid dependence. For example, the therapy monitoring system can monitor a human response to drug therapy indicative of opioid toxicity, opioid withdrawal effects, and/or opioid dependence.

Another exemplary application of the therapy monitoring system includes the monitoring of patients suffering from or undergoing treatment for heart failure. For example, the therapy monitoring system can monitor a human response to drug therapy indicative of toxic inotropic effects, bradycardia, and/or excessive diuresis.

Another exemplary application of the therapy monitoring system includes the monitoring of patients suffering from or undergoing treatment for Alzheimer's disease. For example, the therapy monitoring system can monitor a human response to drug therapy indicative of behavioral changes, falls or increased risk of falls, and/or sleep disturbance.

Another exemplary application of the therapy monitoring system includes the monitoring of patients suffering from or undergoing treatment for Parkinson's disease. For example, the therapy monitoring system can monitor a human response to drug therapy indicative of ON/OFF effects, tremor and/or dyskinetic changes, gait changes, falls or increased risk of falls, and/or sleep disturbance.

Another exemplary application of the therapy monitoring system includes the monitoring of patients suffering from or undergoing treatment for insomnia. For example, the therapy monitoring system can monitor a human response to drug therapy indicative of sleeplessness, excessive sedation, and/or sleep disturbance.

Another exemplary application of the therapy monitoring system includes the monitoring of patients suffering from or undergoing treatment for asthma & chronic obstructive pulmonary disease. For example, the therapy monitoring system can monitor a human response to drug therapy indicative of shortness of breath, wheezing, and/or dry cough.

Another exemplary application of the therapy monitoring system includes the monitoring of patients suffering from or undergoing treatment for multiple sclerosis. For example, the therapy monitoring system can monitor a human response to drug therapy indicative of changes in tremor, changes in gait, and/or falls or increased risk of falls.

### 6 IMPLEMENTATIONS

the approaches described above can be implemented, for example, using a programmable computing system executing suitable software instructions or it can be implemented in suitable hardware such as a field-programmable gate array (FPGA) or in some hybrid form. For example, in a programmed approach the software may include procedures in one or more computer programs that execute on one or more programmed or programmable computing system (which may be of various architectures such as distributed, client/server, or grid) each including at least one processor, at least one data storage system (including volatile and/or non-volatile memory and/or storage elements), at least one user interface (for receiving input using at least one input device or port, and for providing output using at least one output device or port). The software may include one or more modules of a larger program, for example, that provides services related to the design, configuration, and execution of dataflow graphs. The modules of the program (e.g., elements of a dataflow graph) can be implemented as data structures or other organized data conforming to a data model stored in a data repository.

The software may be stored in non-transitory form, such as being embodied in a volatile or non-volatile storage medium, or any other non-transitory medium, using a physical property of the medium (e.g., surface pits and lands, magnetic domains, or electrical charge) for a period of time (e.g., the time between refresh periods of a dynamic memory device such as a dynamic RAM). In preparation for loading the instructions, the software may be provided on a tangible, non-transitory medium, such as a CD-ROM or other computer-readable medium (e.g., readable by a general or special purpose computing system or device), or may be delivered (e.g., encoded in a propagated signal) over a communication medium of a network to a tangible, non-transitory medium of a computing system where it is executed. Some or all of the processing may be performed on a special purpose computer, or using special-purpose hardware, such as coprocessors or field-programmable gate arrays (FPGAs) or dedicated, application-specific integrated circuits (ASICs). The processing may be implemented in a distributed manner in which different parts of the computation specified by the software are performed by different computing elements. Each such computer program is preferably stored on or downloaded to a computer-readable storage medium (e.g., solid state memory or media, or magnetic or optical media) of a storage device accessible by a general or special purpose programmable computer, for configuring and operating the computer when the storage device medium is read by the computer to perform the processing described herein. The inventive system may also be considered to be implemented as a tangible, non-transitory medium, configured with a computer program, where the medium so configured causes a computer to operate in a specific and predefined manner to perform one or more of the processing steps described herein.

A number of embodiments of the invention have been described. Nevertheless, it is to be understood that the foregoing description is intended to illustrate and not to limit the scope of the invention, which is defined by the scope of the following claims. Accordingly, other embodiments are also within the scope of the following claims. For example, various modifications may be made without departing from the scope of the invention. Additionally, some of the steps described above may be order independent, and thus can be performed in an order different from that described.

What is claimed is:

1. A method for monitoring a patient undergoing therapy, the method comprising:
wirelessly tracking a motion of the patient over time to generate a sequence of data representing the motion of the patient over time;
processing the sequence of data representing the motion of the patient over time using a biometric parameter extraction module to extract data representing one or more biometric parameters;
determining an estimated response of the patient to the therapy based at least in part of the data representing one or more biometric parameters, and
providing a characterization of the estimated response of the patient to the therapy to one or more of a caretaker, the patient, or a clinician.

2. The method of claim 1 wherein wirelessly tracking the motion of the patient over time includes tracking the motion of the patent using a radio frequency-based motion tracking system.

3. The method of claim 2 wherein the radio frequency-based motion tracking system includes an FMCW radar motion tracking system.

4. The method of claim 2 wherein the radio frequency-based motion tracking system is configured to emit radio frequency signals and to receive radio frequency signals.

5. The method of claim 1 wherein wireless tracking the motion of the patient over time includes tracking the motion of the patient using reflections of electromagnetic signals from the patient.

6. The method of claim 1 wherein wireless tracking the motion of the patient over time includes tracking the motion of the patient using reflections of ultrasonic signals from the patient.

7. The method of claim 1 wherein extracting one or more biometric parameters from the motion of the patient includes extracting one or more of location, pose, gait, mobility, activity, breathing, heartrate, emotion, and sleep patterns.

8. The method of claim 1 wherein determining the estimated response of the patient to the therapy includes processing the one or more biometric parameters using a model configured to determine the estimated response of the patient to the therapy based on the one or more biometric parameters.

9. The method of claim 8 wherein the model includes an artificial neural network.

10. The method of claim 1 wherein wirelessly tracking the motion of the patient over time includes determining a sequence of observations of a position of the patient over time.

11. The method of claim 10 wherein extracting the one or more biometric parameters from the motion of the patient includes determining a progression the one or more biometric parameters over time.

12. The method of claim 1 wherein determining the estimated response of the patient to the therapy includes determining that the patient has a subtherapeutic response to the therapy.

13. The method of claim 1 wherein determining the estimated response of the patient to the therapy includes determining that the patient has a toxic response to the therapy.

14. The method of claim 1 wherein determining the estimated response of the patient to the therapy includes identifying dose dependent adverse effects to the therapy suffered by the patient.

15. The method of claim 1 wherein determining the estimated response of the patient to the therapy includes identifying withdrawal effects of the therapy suffered by the patient.

16. The method of claim 1 wherein determining the estimated response of the patient to the therapy includes identifying one or more of drug dosing changes or alterations, drug dose increase, drug dose decrease, wearing off of a drug, drug initiation or cessation, drug-drug interactions, drug-food interactions, drug abuse, drug misuse, and illicit drug use.

17. The method of claim 1 further comprising determining a compliance to the therapy by the patient based at least in part on the estimated response of the patient to the therapy.

18. The method of claim 1 wherein the therapy includes a single or multiple drug therapy.

19. A system for monitoring a patient undergoing therapy, the system comprising:
a wireless sensor for wirelessly tracking a motion of the patient over time to generate a sequence of data representing the motion of the patient over time;
a biometric parameter extraction module for processing the sequence of data representing the motion of the patient over time to extract data representing one or more biometric parameters;
a therapy response model for determining an estimated response of the patient to the therapy based at least in part of the data representing one or more biometric parameters, and
an output providing a characterization of the estimated response of the patient to the therapy to one or more of a caretaker, the patient, or a clinician.

20. Software stored in non-transitory form on a machine-readable medium, for monitoring a patient undergoing therapy, the software including instructions for causing a computing system to:
wirelessly track a motion of the patient over time to generate a sequence of data representing the motion of the patient over time;
process the sequence of data representing the motion of the patient over time using a biometric parameter extraction module to extract data representing one or more biometric parameters;
determine an estimated response of the patient to the therapy based at least in part of the data representing one or more biometric parameters, and
provide a characterization of the estimated response of the patient to the therapy to one or more of a caretaker, the patient, or a clinician.
